(19) United States
(12) Patent Application Publication
(10) Pub. No.: US 2012/0207718 A1
(43) Pub. Date: Aug. 16, 2012

(54) THIN SHELL GRAFT FOR CARTILAGE RESURFACING

(76) Inventors: KEVIN R. STONE, San Francisco, CA (US); THOMAS J. TUREK, Pacifica, CA (US)

(21) Appl. No.: 13/397,540

(22) Filed: Feb. 15, 2012

Related U.S. Application Data
(60) Provisional application No. 61/443,665, filed on Feb. 16, 2011.
(60) Provisional application No. 61/499,288, filed on Jun. 21, 2011.

(51) Int. Cl. A61F 2/08; A61P 19/00; A61K 35/32

(52) U.S. Cl. 424/93.7; 623/14.12

(57) ABSTRACT

An articular cartilage shell graft is designed to treat the arthritic population for which current biologic treatments are insufficient and as a biological-stage repair for intervention before prosthetic knee arthroplasty.

### FIELD

The present invention relates to anatomic cartilage resurfacing with intact hyaline cartilage and subchondral bone for the treatment of joint pathologies, such as osteoarthritis.

### BACKGROUND

Current standard of care for massive cartilage defects or degeneration of femoral condyles is either total knee replacement or unicondylar knee replacement. Although these prosthetic knee reconstructions reduce pain and restore some function, they severely limit a patient's range of activities, and as mechanical synthetic devices have limited functional life spans.

Arthritis is the leading cause of disability in the United States, limiting the everyday activities of more than 70 million Americans and resulting in over 300,000 artificial joint implants annually. If non-operative or arthroscopic treatment fails, the current standard of care for moderate to severe knee arthritis is prosthetic total knee arthroplasty (TKA) or unicompartmental knee arthroplasty (UKA). Although TKA and UKA are often effective at eliminating pain, strenuous and high impact activities can cause damage to the implant components and the surrounding bone. In addition, prosthetic implants are truly an end-stage solution and do not spare the normal biology or anatomy of the knee. Hence, prosthetic implant surgeries are not often recommended for young or high-demand patients.

In efforts to repair rather than replace damaged knee cartilage with end-stage metal and plastic, there has been a dedicated focus to develop biologic techniques for the treatment of knee cartilage damage. Several biologic treatments have been developed in the past two decades and included: debridement/chondroplasty; microfracture; autologous chondrocyte implantation (ACI and MACI); Osteochondral Autograft Transfer (OATS)/Mosaicplasty; and Articular Cartilage Paste Grafting.

Existing surgical techniques produce variable degrees of success clinically and are limited to focal, or small to moderately sized, lesions in articular cartilage. Treatment becomes more difficult as the arthritic lesions increase in size, as is the case in severe arthritis. Although articular cartilage paste grafting has been successfully used in arthritic knees, the technique is still limited medium size defects not involving the entire condyle. Articular cartilage shell grafting is designed to treat large cartilage surfaces in arthritic knees.

### SUMMARY

Allogeneic fresh harvested osteochondral dowels or shell grafts have been used to restore degenerated articular surfaces of the femoral condyle. In combination with point of use derived stem cells, or culture expanded cells, or autologous factors, or exogenous bio-active agents may prove to have expanded utility in the prevention or delaying of knee replacement procedures. The combination of matrices and biological agents, either proteins or cells, has shown utility for tissue regeneration in orthopaedic indications. As described herein, the binary application of cells and/or bio-active agents and immunochemically modified and sterilized xenograft or allogenieic cartilage shell graft has advantages over current cartilage resurfacing techniques. These advantages include application in severely arthritic knees, and utility for repair of massive defects and immediate surface integrity at an earlier time point postoperatively.

An articular cartilage shell graft is designed to treat the arthritic population for which current biologic treatments are insufficient and as a biological-stage repair for intervention before prosthetic knee arthroplasty. An exemplary shell graft/cell isolate device is intended for use in symptomatic, Grade IV, predominantly unicondylar knee arthritis where a biological repair is preferred. Indications include young and higher demand patients where an end-stage prosthetic replacement may have insufficient life-span.

Described herein is a graft for implantation in an articular cartilage defect in a bearing region of an articular surface of a joint of a patient, wherein the articular cartilage defect is characterized by a base surface disposed about a defect axis extending substantially normal to the articular surface at the defect, and defined by a defect base periphery and having lateral surfaces extending in the direction of the graft axis from the defect base periphery with monotonically increasing radii with respect to the defect axis. The graft comprises an intact tissue block extending along a graft axis from an outer surface at an outer end to an inner surface at an inner end. The outer surface is bounded by an outer end periphery and extends transverse to the graft axis at the outer end, and the inner surface is bounded by an inner end periphery and extends transverse to the graft axis at the inner end. The graft has a lateral surface extending along and about the graft axis from the outer end periphery to the inner end periphery, and includes at the outer end, hyaline cartilage extending from the outer surface and in the direction of the graft axis, toward the inner end. The graft further includes at the inner end, subchondral bone extending from the inner surface and in the direction of the graft axis, toward the outer end. The outer surface as defined by the outer end periphery, and has a shape adapted to overlie and extend beyond the bearing region of the articular surface of a joint when the graft axis is substantially coaxial with the defect axis.

The graft, or tissue block, is further described as having an inner surface as defined by the inner end periphery, and having a shape adapted to overlie and is coextensive with the base surface of the defect when the graft axis is substantially coaxial with the defect axis. The lateral surface of the graft is substantially complementary to the lateral surface of the defect. The thickness T of the graft in the direction of the graft axis, is such that when implanted, the graft is resistant to fracture under anatomical load of the patient.

In an embodiment, the patient is a human, the tissue block is from a human, and T is in the approximate range 2.5-12 mm. The tissue block is substantially void of cellular activity, has reduced cellular activity, or has near-normal cellular activity.

In an embodiment, the tissue block is sterilized, such as by supercritical CO₂ sterilization or ionizing radiation, to effect a bioburden reduction of at least 10⁶. In an embodiment, the subchondral bone of the tissue block is infused with exogenous cells, such as by vacuum-infusion. Alternatively, the tissue block is infused with one or more bio-active agents or factors to enhance healing, such as by vacuum-infusion with one or more factors to enhance healing.

In an embodiment, the tissue block includes distributed therein, a cell population including one or more cells from the group consisting of adult or embryonic mesenchymal stem cells, embryonic stem cells, fibroblasts, chorndrocytes, chondroblasts, pro-chondroblasts, osteocytes, synoviocytes, osteoclasts, pro-osteoblasts, monocytes, pro-cardiomyocytes, pericytes, cardiomyoblasts, cardiomyocytes, myocytes or combinations thereof. Alternatively, the cell population includes cells from bone marrow, cells from adipose tissue, and cells from plasma derived fractions of autologous blood. In an embodiment, at least a portion of the cell population is vacuum-infused into the tissue block.

In an embodiment of the thin shell graft, a loading ratio of cells of the population in a volume of cells to volume of graft, ranges from about 1:3 to 3:1. In yet another embodiment, the tissue block includes distributed therein one or more bioactive agents. These bioactive agents include one or more from the group consisting of fibroblast growth factors, epidermal growth factors, kertinocyte growth factors, vascular endothelial growth factors, platelet derived growth factors, transforming growth factors, bone morphogenic proteins, parathyroid hormone, calcitonin, prostaglandins, ascorbic acid, and combinations thereof.

In another embodiment, a loading ratio of cells of bioactive agents in a volume of cells to volume of graft, ranges from about 1:3 to 3:1. Further, an embodiment of the tissue block is from an animal from the group consisting of porcine, bovine, equine, or ovine animals, which may further be pursuant to de-antigenation by removal of alpha-galactosyl epitopes with glycosidase. Alternatively, the tissue block is from a human. Specifically, the articular surface is a joint from the group consisting of knee, jaw, shoulder, elbow and hip.

Also disclosed herein is a method for infusing a cell population or one or more bioactive agents into a tissue block extending from a first end to a second end opposite thereto, and including at the first end, hyaline cartilage extending from the first end and toward the second end, and including at the second end, subchondral bone extending from the second end toward the first end. The method comprises the steps of: A) positioning the cell population or bioactive agents onto at least on surface of the tissue block; and B) applying a pressure gradient to the tissue block; having the cell population or bioactive agents thereon. The application of the pressure gradient comprises the steps of applying a pulsed vacuum sequence to the tissue block having the cell population or bioactive agents thereon, cycling n times between approximately 0 mmHg (ambient) and approximately 750 mmHg, for duration m minutes, where n and m are integers.

In an embodiment, the portions of cycles are uniform from cycle to cycle, and m is in the range of about 3-10 cycles and n is in the range of about 1 to 3 minutes. In another embodiment, following the application of the pulsed vacuum sequence to the tissue block, the graft is incubated under vacuum for a period T₀ at a pressure P. In alternative embodiments, T₀ is in the range of about 45-120 minutes and P is in the range of about 200-750 mmHg, and preferably, T₀ is in the range of about 45-120 minutes and P is in the range of about 300-550 mmHg. In other embodiments, the pressures and the durations of the respective portions of the cycling may differ, although in the preferred ranges, in a given process.

Also disclosed herein is a method for preparing a human allograft or xenograft for implantation in an articular cartilage defect. The method comprises the steps of: A) asceptically harvesting a graft including an intact tissue block from a host, wherein the tissue block: a) extends along a graft axis from an outer surface at an outer end to an inner surface at an inner end, wherein the outer surface is bounded by an outer end periphery and extends transverse to the graft axis at the outer end, and the inner surface is bounded by an inner end periphery and extends transverse to the graft axis at the inner end; b) has a lateral surface extending along and about the graft axis from the outer end periphery to the inner end periphery; c) has at the outer end, hyaline cartilage extending from the outer surface and in the direction of the graft axis, toward the inner end; and d) has at the inner end, subchondral bone extending from the inner surface and in the direction of the graft axis, toward the outer end. The method further comprises the steps of: decellularizing the graft; de-antigenizing the graft; sterilizing the graft; and infusing a cell population or one or more bioactive agents into a tissue block if the graft.

Also disclosed herein is a method for implanting a graft in an articular cartilage defect in a bearing region of a articular surface of a joint of a patient. The method comprises the steps of: A) preparing the articular cartilage defect whereby it is characterized by a base surface disposed about a defect axis extending substantially normal to the articular surface at the defect, and defined by a defect base periphery and having a lateral surface extending in the direction of the graft axis from the defect base periphery with monotonically increasing radii with respect to the defect axis; and B) preparing a thin shell graft as described in further detail herein. In an embodiment of this method, the outer surface as defined by the outer end periphery, has a shape adapted to overlie and extend beyond the bearing region of the articular surface of a joint when the graft axis is substantially coaxial with the defect axis. Further, the inner surface as defined by the inner end periphery, has a shape adapted to overlie and is coextensive with the base surface of the defect when the graft axis is substantially coaxial with the defect axis. The lateral surface of the graft is substantially complementary to the lateral surface of the defect, and the maximum thickness T of the graft in the direction of the graft axis, is such that when implanted, the graft is resistant to fracture under anatomical load of the patient. The method further comprises the additional steps of: preparing the lateral surface of the defect for receipt of the graft by the step of morselizing the lateral wall and the base surface through a subchondral plate underlying the defect; applying the graft to the defect whereby the lateral surface of the graft is in intimate contact with the lateral surface of the defect; and attaching the graft to the base surface of the defect.

The thin shell graft and methods are further defined in the description below, and the appended claims.

### BRIEF DESCRIPTION OF THE FIGURES

FIG. 1A is a perspective view of an exemplary shell graft.

FIG. 1B is a cross-section of the device of FIG. 1A along a principle plane.

FIG. 1C is a perspective view of the shell graft of FIG. 1A positioned over the defect in which the graft is to be implanted.

FIG. 1D is a cross-section along a principle plane of the device of FIG. 1C as implanted in the defect shown in FIG. 1C.

FIG. 2A is a top view diagram of a thin shell graft intended for condyle reconstruction.

FIG. 2B is a side view diagram of the thin shell graft of FIG. 2A.

FIG. 2C is a 10× magnified side view diagram of the thin shell graft of FIG. 2A, showing pre-processing with articular surface (left) and subchondral bone (right).

FIG. 2D is a 10×magnified side view diagram of the thin shell graft of FIG. 2A, post-processing, showing intact construct morphology essentially devoid of cells.

FIG. 3A is a side plan view of an embodiment of the thin shell graft.

FIG. 3B is a top plan view of an embodiment of the thin shell graft.

FIG. 4 is a flowchart for an embodiment of the process for preparing and implanting a minimally-processed or unprocessed human allograft thin shell graft.

FIG. 5A is a perspective view of condyle bone, showing morselization of a condyle lesion.

FIG. 5B is a perspective view of the same bone of FIG. 5A, showing placement of a thin shell graft.

FIG. 5C is a perspective view of the same bone of FIG. 5A and FIG. 5B, showing a thin shell graft in position over the morselized portion of the bone, showing peripheral fixation of the device.

FIG. 6 is flowchart for an embodiment of the process for preparing and implanting a processed human allograft thin shell graft.

FIG. 7 is a composite graph showing ICRS cartilage repair assessment scoring.

### DETAILED DESCRIPTION

The disclosed shell graft/cell isolate device is a binary device with two primary components: (1) a shell graft; and (2) cell isolate.

There are two sources of cells that are preferred for the present shell graft: i) point of service isolated autologous adult mesenchymal stem cells; and ii) culture expanded adult mesenchymal stem cells. Autologous point of service stem cells are harvested and isolated from marrow or adipose tissue, enriched, and incubated with the present shell graft in situ at the time of indexed cartilage repair surgery. An alternate path utilizes culture expanded marrow-derived cells that are harvested from marrow aspirate. Culture expanded stem cells can be isolated from aspirate, either adipocyte or marrow, or blood and expanded before implantation. Allogeneic adult mesenchymal stem cells are an alternate to autologous stem cells. These cells are isolated and differentiated from donors and are commercially available in standardized batches.

Other embodiments include use of mixed cell populations from either autologous or allogeneic source.

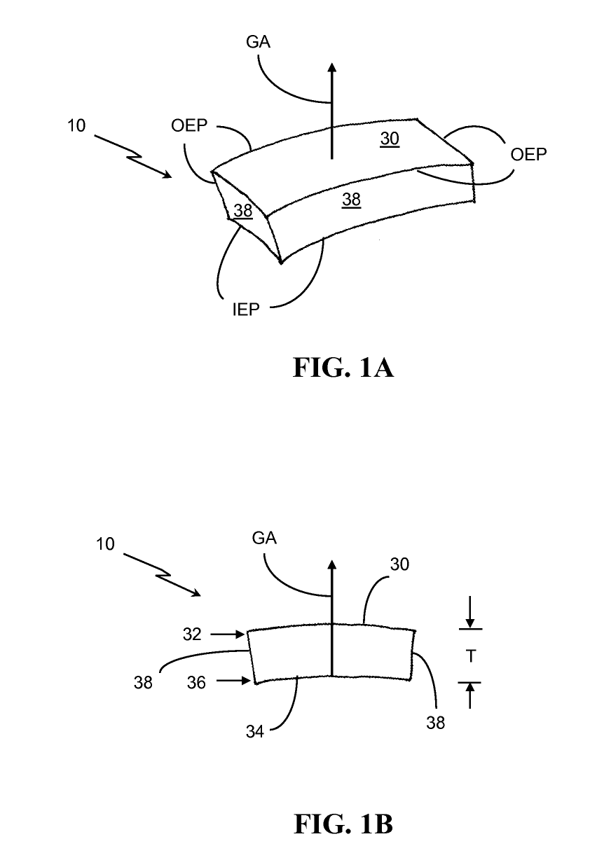

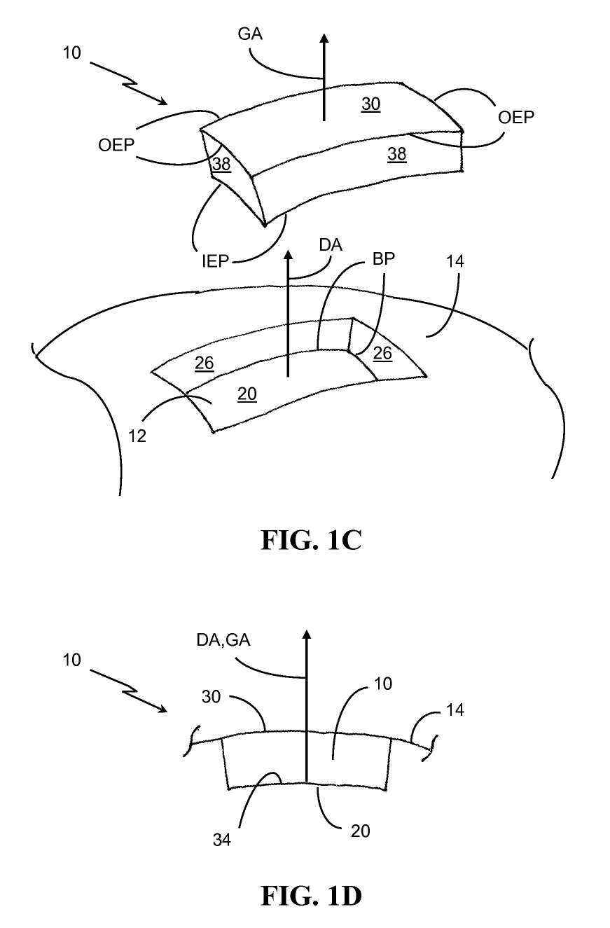

An exemplary graft 10 is shown in FIGS. 1A-1D. The graft 10 is adapted for implantation in an articular cartilage defect 12 in a bearing region of an articular surface 14 of a joint of a patient. The surface 14 may be a weight-bearing area, for example a condylar surface, or may be a surface over which a tendon passes in normal articular motion, exhibiting sheer forces on the surface.

As illustrated in FIG. 1C, an articular cartilage defect 12 is characterized by a base surface 20 disposed about a defect axis DA extending substantially normal to the articular surface 14 at the defect. The base surface 20 of articular cartilage defect 12 is bounded by a defect base periphery, part of which is shown in FIG. 1C and identified by reference designation BP. The defect 12 is further characterized by lateral surfaces 26 (two of which are shown in FIG. 1C) extending from the defect base periphery 20, with monotonically increasing radii with respect to the defect axis DA.

Preferably, for implantation with the illustrated graft 10, the particular cartilage defect 12 is a naturally-occurring defect which has been machined, or otherwise shaped to have the above-noted geometrical characteristics.

The illustrated graft 10, preferably is an intact tissue block as identified in FIG. 1A by reference numeral 10. The tissue block 10 extends along a graft axis GA from an outer surface 30 at an outer end 32, to an inner surface 34 at an inner end 36. The outer surface 30 is bounded by an outer end periphery OEP (four segments of which are shown in FIGS. 1A and 1C). The inner surface 34 is bounded by and inner end periphery IEP (two segments of which are shown in FIGS. 1A and 1C).

The tissue block 10 has lateral surfaces 38 (two of which are shown in FIGS. 1A, 1B and 1C), extending along and about the graft axis GA, from the outer end periphery OEP to the inner end periphery IEP.

The tissue block 10 includes at the outer end 32, hyaline cartilage extending from the outer surface 30 and in the direction of the graft axis GA from the outer end periphery OEP toward the inner end.

The tissue block 10 includes at the inner end 36, subchondral bone extending from the inner surface 34 and in the direction of the graft axis GA from the inner end perforation IEP toward the outer end 32.

The outer surface 30 of tissue block 10, as defined by the outer end periphery OEP, has a shape adapted to overlie and extend beyond the bearing region of the articular surface 14 of a joint when the graft axis GA is substantially coaxial with the defect axis DA, that is, as shown in FIGS. 1C and 1D.

The inner surface 34 of tissue block 10, as defined by the inner end periphery IEP, has a shape adapted to overlie the base surface 20 the articular surface 14 of a joint when the graft axis GA is substantially coaxial with the defect axis DA, that is, as shown in FIGS. 1C and 1D.

The lateral surface 38 of the tissue block 10, as a shape which is substantially complementary to the lateral surface 26 of the defect 12.

The thickness T (shown in FIG. 1A) of the tissue block 10 in the direction of the graft axis GA, is such that, when implanted, the tissue block 10 is resistant to fracture under anatomic load.

In FIG. 1D, the tissue block 10 is shown seated in the defect 12, with the lateral surface 38 of tissue block 10 in substantial intimate contact with the lateral surface 26 of the defect, and the inner end surface 34 of the tissue block 10 with substantial intimate contact with the base surface 20 of the defect.

Because the tissue block 10 has as a geometry such that its boundary, defined in part by outer and periphery OEP, extends beyond the bearing region of the articular surface of the joint, the stability of the tissue block 10 in the defect 12 is substantially enhanced, and minimally affected by anatomical motion, for example by movement of opposing bones in a joint. The ability to maintain the border/interface of the tissue block 10 with the boundaries of the defect 12, as well as the maintenance of the lateral surface of tissue block 10 in substantial intimate contact with the lateral surface of defect 12, are very in important in promoting the healing, and integration of the tissues of the tissue block 10 and the tissue bordering defect 12.

Further, the composition of the tissue block 10, including hyaline cartilage at the inner end 32 and subchondral bone at the outer end 36, with the transition from hyaline cartilage to subchondral bone occurring between those ends 32 and 36, provides a structure amenable to integration of the tissues of the tissue block 10 and the defect 12. Further, the thickness T of the tissue block 10 is determined, such that anatomical motion minimally affects the stability, being resistant to fracture under anatomical load. For a tissue block to be of optimal usage for implantation into a defect of a human joint, the tissue block thickness T is preferably in the approximate range 2.5-12.0 mm. For other animals, the tissue block thickness T is determined based on the size and weight, and load characteristics for those animals, considering both the source animal and the recipient of the graft.

In one form, the tissue block 10 is substantially void of cellular activity, for example as is usual when porcine tissue used as a xenograft for a human. In such cases, the porcine tissue can be freeze/thaw treated, one or more times, to substantially “kill” all cells in the tissue block, and is further treated with alpha-galactosidase to remove alpha-gal epitopes (substantially removing the immunogenic effect of the porcine tissue).

In another form, the intact tissue block is characterized by near normal cellular activity, for example in cases where the tissue block is an allograft, and there is a negligible immune response issue.

In yet other forms, the intact tissue block is characterized by reduced cellular activity.

In general, the tissue block is sterilized, prior to use. The sterilization may be provided by a supercritical CO₂ sterilization process. Alternatively, or in addition, sterilization may be provided by radiation. Preferably, sterilization is provided to effect a bioburden reduction of at least 10⁶.

Further, the tissue block 10 is infused with one or more of exogenous cells, and/or bio-active agents or factors, such as growth factors, to enhance healing. Preferably such elements are introduced by a vacuum-infusion process.

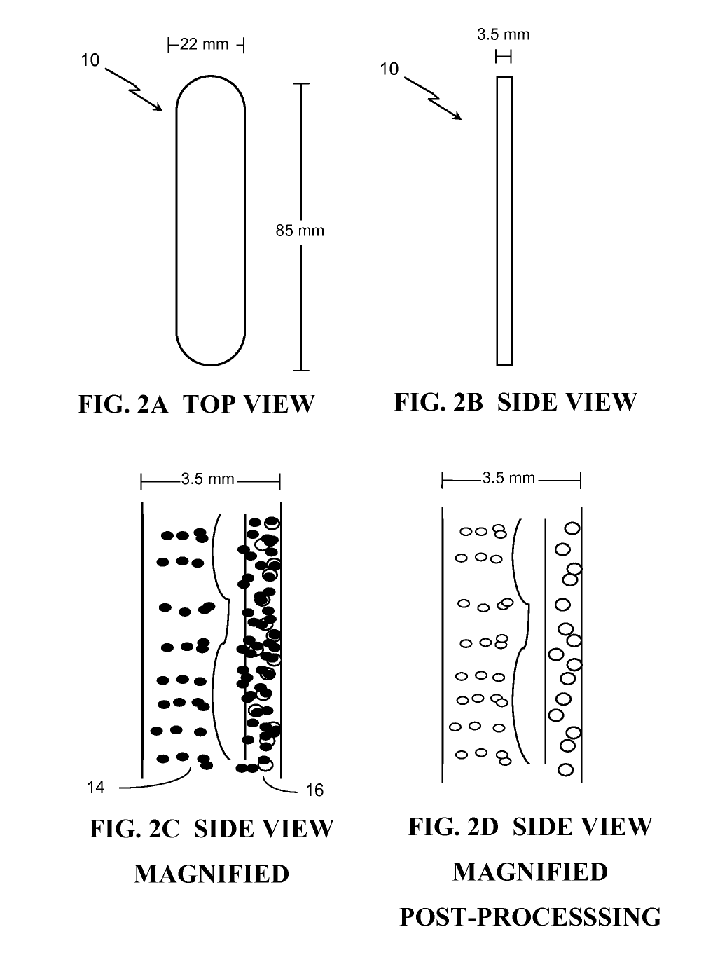

In an embodiment, the thin shell graft device 10, is a modified and sterilized porcine or allograft matrix composed of trochlear cartilage and subchondral bone. In a preferred form, and as illustrated in FIGS. 2A-2D, processing includes decellularization of an intact tissue block 10, precision machining to a nominal depth, or thickness, of 3.5-mm (range 2 to 5 mm), width of 22 mm (range 15 to 35 mm), and length of 85 mm (range 40 to 100 mm), together with terminal sterilization. The device 10 is preferably supplied sterile and frozen, for one time use. FIGS. 2A-2D show examples of the present thin shell graft 10 as intended for condyle reconstruction. FIG. 2A shows a top view of the thin shell graft 10; FIG. 2B shows the same graft 10 in a side view. FIG. 2C is a 10× magnified view of the side view of FIG. 2B, showing the articular surface 14 and the subchondral bone 16. FIG. 2D shows the same side view of FIG. 2C, post-processing, illustrating intact construct morphology of the graft 10 essentially devoid of cells, as discussed in further detail below.

In an embodiment, a thin shell graft 10 is aseptically harvested from articular cartilage from mammalian species including, but not limited to human, bovine, equine, porcine, ovine and nubine sources. The graft 10 is composed of hyaline cartilage and underlying subchondral bone in vertical axis. Transverse axis dimensions are of sufficient size to cover and, preferably, extend beyond the bearing region of intended cartilage reconstruction surface.

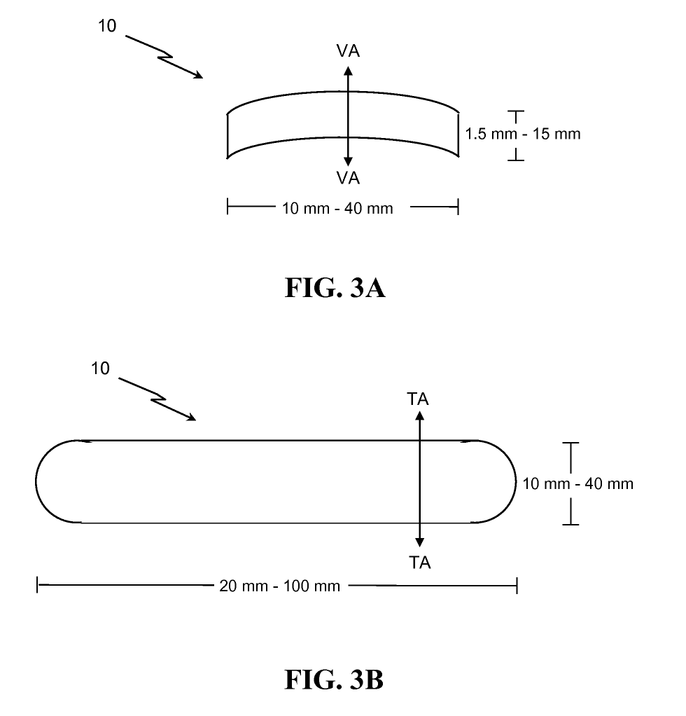

As illustrated in FIG. 3, the vertical axis VA of the graft 10 can range from 1.5 mm to 15 mm, preferably 2.5 mm to 12 mm. The transverse dimension, along the transverse axis TA can vary depending on the intended reconstruction site, and can range from 10 mm to 40 mm in width and 20 mm to 100 mm in length. Preferable dimensions for condylar reconstuction are 15 mm to 35 mm in width and 40 mm to 100 mm in length.

The harvested graft is precision machined, either manually or with robotic assistance, with dimensions corresponding a complimentary, same- sized, machined (preferably at the time of surgical implantation) defect in the intended recipient reconstruction area.

In alternative embodiments, harvested and sized grafts are either minimally processed (as for human allografts), or processed (as for xenografts or processed human allografts). Minimal processing preferably is used for fresh harvested human grafts, stored short-term in media, to retain cellular activity until implantation.

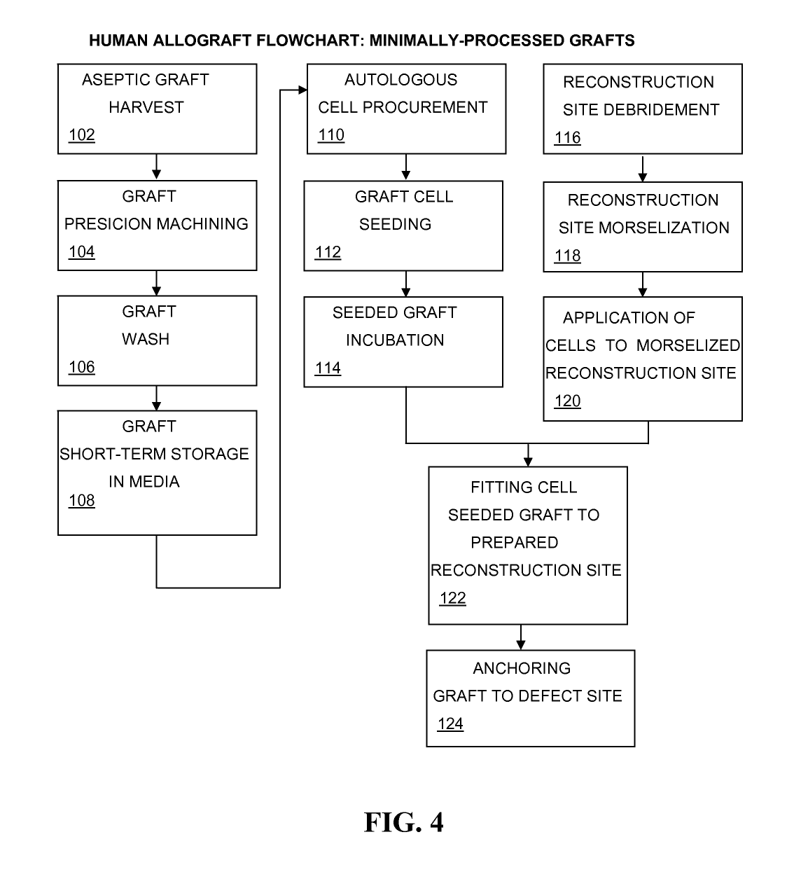

As shown in the flowchart of FIG. 4, for minimally processed human allografts, after aseptic harvest and sizing 102, the grafts are washed 106 with standard physiologic solutions, transferred to standard growth media with antibiotics 108 and stored at 4° C. until implantation. The physiologic solutions, standard growth media (such as MEM), and the selected antibiotics all are known to those skilled in the art. All are generally commercially available. The specific formulation and compositions will depend on the origin of the cells and the protocol used for the harvest.

In an embodiment of the thin shell graft and process, autologous cells are procured 110. The graft is seeded 112 with purified or mixed cell populations including, but not limited to, adult or embryonic mesenchymal stem cells, embryonic stem cells, fibroblasts, chondrocytes, chondroblasts, pro-chondroblasts, osteocytes, synoviocytes, osteoblasts, pro-osteoblasts, monocytes, pro-cardiomyocytes, pericytes, cardiomyoblasts, cardiomyocytes, myocytes or multiple combinations of the above from bone marrow or adipose derived stem cells, and/or plasma derived fractions of autologous blood.

In another embodiment, the thin shell graft is seeded with biological agents including, but not limited to fibroblast growth factors, epidermal growth factors, keratinocyte growth factors, vascular endothelial growth factors, platelet derived growth factors, transforming growth factors, bone morphogenic proteins, parathyroid hormone, calcitonin, prostaglandins, ascorbic acid. or multiple combinations of the above.

In the embodiment having cell seeded graft devices, the cells or bio-active agents are loaded in a volume of cells/ bioactive agent to volume of graft in a range of ratios of from about 1:3 to 3:1, preferably a range of from about 1:1 to 3:1. Such loading of the cells/bioactive agents is static, followed by incubation 114 under culture conditions for hours or days before implantation. Such loading is facilitated with a pressure gradient at ambient and/or physiological temperature prior to implantation.

In an embodiment, seeding 112 is achieved using a pulsed vacuum sequence cycled 3 to 10 times from 0 mmHg (ambient) to 750 mmHg, preferably 0 mmHg to 550 mmHg in 1 to 3 minute cycles. Incubation 114 is performed under vacuum from 200 to 750 mmHg, preferably 300 to 550 mmHg, for 45 minutes to 120 minutes.

The purpose of this processing is to affect a three-dimensional distribution of cells throughout the thin shell grafts. Pressure differential drives the biological agent-containing solution into the shell graft structure, displacing entrapped air, and allowing penetration throughout the interstices of the graft, allowing such cells/bio-active agents to bind to mineral and collagen components within the graft. Further incubation allows for continued binding of cells and biological agents before implantation.

The process shown in FIG. 4 further involves debriding 116 the surgical implant site, and sizing the site complementary to the processed graft device. After morselizing the defect bed 118, an aliquot of cells or bioactive agent is applied to the surgical implant site 120. The implant site bed is morselized with perforations through the subchondral plate and accessing marrow. These perforations allow endogenous cells and bioactive factors from the host to percolate through the implant bed to the graft interface. These endogenous mobilized cells and factors contribute to interfacial healing process and are instructive to exogenous cells and factors added to the implant site and graft.

Once the shell graft is harvested and prepared, it is implanted into the desired site 122, and the graft device is anchored to the site 124.

An embodiment of the process for implanting the shell graft involves the following steps:

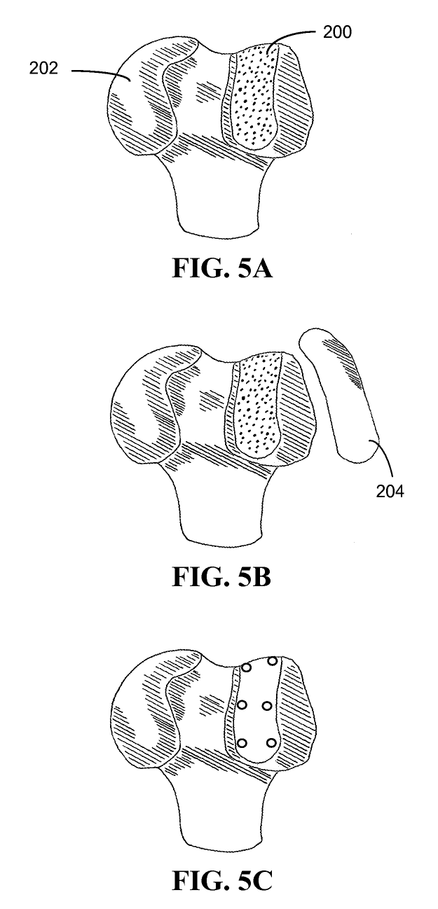

Affected condyle is debrided and lesion bed morselized through to subchondral bone. FIG. 5A shows a morselized lesion bed 200 of a condyle bone 202.

As shown in FIG. 5B, a seeded shell graft 204 is brought to the surgical field and sized to the prepared lesion bed dimensions.

During the surgical procedure, the device is passed through an anterior arthroscopic portal.

The graft device 204 is positioned and secured using resorbable anchors through the graft, as shown in FIG. 5C.

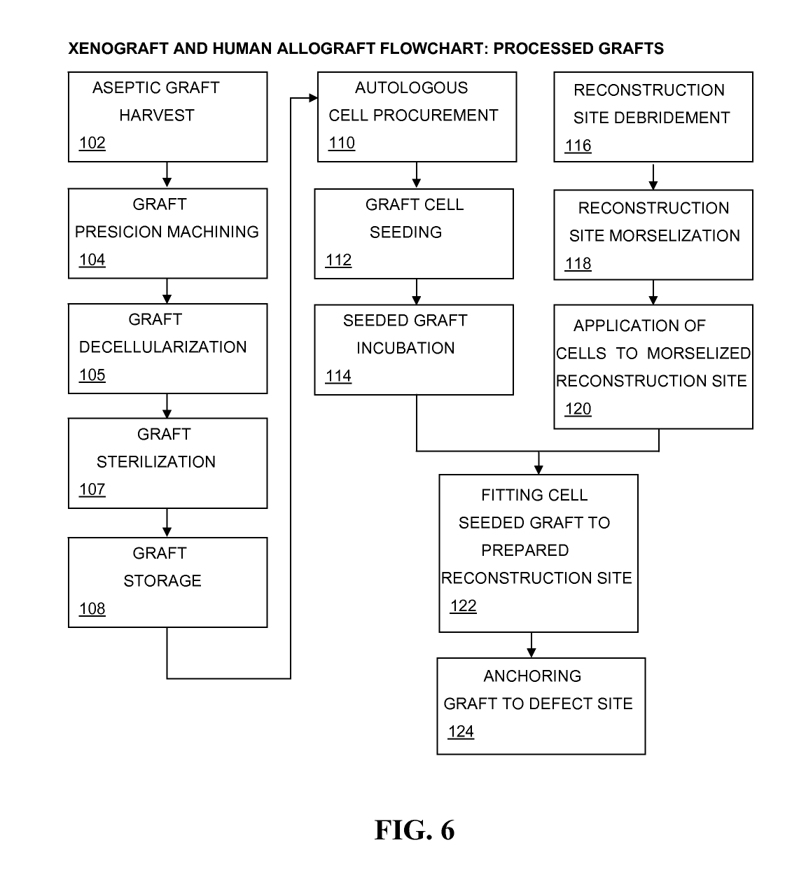

A process for preparing the graft device and implanting it at the desired site is shown in the flowchart of FIG. 6, for processed human allografts and xenografts. After aseptic harvest and sizing 102, the grafts are subject to a series of steps, in no specific order, of decellularization 105, and/or de-antigenation, and sterilization 108. As used here, the term decellularization means wherein cellular inactivation and decellularization of allografts or xenografts is accomplished by treatment with one or more of freeze/thaw cycling, hypotonic/hypertonic solutions, ionic/anionic detergents, compressed CO₂ gas-facilitated lavage. The purpose of this processing is to substantially reduce cell content, cellular debris content, and cellular biological activity.

As used herein, the term de-antigenation means wherein the xenograft is made essentially devoid of alpha-galactosyl epitope using glycosidase enzyme. The purpose of this processing is to substantially reduce the antigenicity of the graft, to yield a graft with preferable immunocompatibility for intended implantation in humans.

As used herein, the term sterilization means wherein the allograft or xenograft is sterilized using ionizing radiation or super-critical CO₂ sterilization or a combination thereof. The purpose of this processing is to provide a sterile device, with sterilization compliant to current device standards, as a preferred graft intended for implantation in humans.

Grafts can be prepared for final storage in a physiologic solution or lyophilized. Grafts stored in solution can be stored below ambient temperature, preferably frozen storage at −20 to −80° C. Lyophilized grafts are stage-dried to a final moisture level less than 10%. Lyophilized grafts can be stored at ambient temperature, preferably below ambient temperature with storage at 4° C. to −20° C.

Following the steps of graft harvest 102, decelluarization 105, and/or graft sterilization 107, and graft storage 108, the process for seeding the graft device 112 through anchoring the graft to the prepared site 124 is essentially the same as that used for minimally-processed or unprocessed graft devices, as described above and in FIG. 4.

An exemplary shell graft/cell isolate kit includes the following components: (a) sterilized shell graft; (b) 2-cc of Stem Cell Isolate (supplied at >10⁵ cells/mL); (c) 3-cc syringe; (d) 18 G 1^{1/2} needle; (e) graft incubation chamber; and (f) vacuum incubator.

In an embodiment, the shell graft/cell isolate device is prepared in the operating room on a sterile table in accordance with the following steps:

2 cc of stem cells are removed from a vial with a 3 cc syringe and 18 G 1^{1/2} needle.

The shell graft is placed in an incubation chamber and cells from the syringe are uniformly expressed over the shell graft. The volume of cells is apportioned to completely cover the graft at a graft volume to cell suspension volume ratio of from about 3:1 to 1:3.

In an embodiment, the shell graft device is placed in a vacuum incubator at 37° C. Stage I is a pulsed vacuum sequence from ambient pressure to 550 mmHg over 1 minute and cycled six times, Stage II is a 45 to 120 minute incubation either under 550 mmHg or at ambient pressure. Total preparation time is in a range of about 60 to 120 minutes.

The cell seeded graft is kept in the incubator until surgical implantation to enhance cell attachment.

In an embodiment, the surgical approach to harvest and device implantation is performed using standard arthroscopic technique.

The thin skin graft and procedure for delivering the same, is further described in the following examples.

### EXAMPLE 1

The in vitro viability of the combination of thin shell graft and culture-expanded bone marrow-derived stem cells (BM-MSCs) is important. The following example identifies cell loading under defined vacuum seeding conditions and short-term culture.

### Materials and Methods

General Thin Shell Graft Device Fabrication. Articular cartilage with underlying subchondral bone was harvested in sheets from trochlear cartilage of adult pigs. A combination of sagittal saws and hand piece saws were used to uniformly size the grafts to a nominal thickness of about 2.0 to 3.5-mm. Post-harvest, the grafts were pulse lavaged with WFI, followed by WFI with 0.5M NaCl and 0.3% Triton X-100. Four rinses in excess PBS were used to washout detergent followed by 1 rinse in glycine/PBS. A glycine/PBS, 2M propylene glycol buffer with bacitracin buffer was used for test article storage and freezing. Materials were stored frozen on dry ice.

Bone Marrow-derived Mesenchymal Stem Cell (MSCs) Preparation. Bone marrow (BM) aspirates were collected from normal volunteer donors after informed consent obtained under an IRB-approved protocol. Isolation of MSCs was performed following standard published protocols. Briefly, MSCs were separated from other BM components by a standard percoll gradient. Cells were expanded in maintenance medium (DMEM-LG+10% selected fetal bovine serum-FBS) for 2 weeks with medium change twice per week, until reaching subconfluence. For these experiments, second passaged cells were used.

Thin Shell Graft and MSC composite loading

After thawing, thin shell grafts were washed three times with phosphate buffer saline as a pre-equilibration step. Test article for in vitro testing was made using a biopsy punch producing 3mm diameter plugs with proximal articular cartilage and distal subchondral bone ends. Thirty microliters (μL) of cell suspension at 0, 10, 40 or 80 million cells per mL in DMEM-LG+10% FBS was placed in a 14 ml polystyrene, round bottom tube with the test article. Cells were loaded into the test article by three cycles of pulse vacuum to 30 mmHg. After vacuum seeding, cell constructs were incubated at 37° C./5% CO₂ for 3 hours to allow for cell attachment. After that, loaded test articles were kept in 24 multiwell plates for medium change (every day for the first week and then every other day for the remainder of the experiments).

### Cell Loading and Short-Term Culture

After cell loading, graft plugs with at 0, 10, 40 or 80 million of Vybrant®—labeled cells per mL and incubation for 3 hrs, seeded constructs were either used as T=0 loading controls or subjected to culture for 5 days in maintenance medium (DMEM-LG+10% selected fetal bovine serum-FBS) to assess initial cell engraftment to the cartilage matrix. Table 1 below outlines the experimental groups tested to identify cell loading parameters and initial cell viability in combination with the thin shell graft.

TABLE 1
Cell Loading and Short-Term Culture

| | CELL DENSITY |
|---|---|
| GROUP #/N | (10⁶ cells/mL) |
| 0 DAYS (loading control) | |
| GRP1: N = 3 | 0 (control) |
| GRP2: N = 3 | 10 |
| GRP3: N = 3 | 40 |
| GRP4: N = 3 | 80 |
| 5 DAYS | |
| GRP5: N = 3 | 0 (control) |
| GRP6: N = 3 | 10 |
| GRP7: N = 3 | 40 |
| GRP8: N = 3 | 80 |

### Results

Table 2 reviews observations on cell loading.

TABLE 2
Cell Loading

| | CELL DENSITY | OBSERVATIONS |
|---|---|---|
| GROUP #/N | (10⁶ cells/mL) | 5 DAYS (in culture) |
| 5 DAYS | | |
| GRP5: N = 3 | 0 (control) | Some signal at periphery within |
| | | articular cartilage |
| GRP6: N = 3 | 10 | 10% of lacunae with cells, few |
| | | cells within trabeculae |
| GRP7: N = 3 | 40 | 40-50% of lacunae with cells, |
| | | clumps of cells within trabeculae |
| GRP8: N = 3 | 80 | 70-75% of lacunae with cells, 3x |
| | | cells within trabeculae as |
| | | compared to 40 × 10⁶ loading |

### Conclusions

Cell Loading and Short-Term Culture. Minimal residual cellular debris left post-graft processing. 80×10⁶ cell loading shows a definite dose response in loading as compared to 40×10⁶ and 10×10⁶ loading. Loaded hMSCs dose-dependently penetrate inside the cartilage ECM with a high percentage of lacunae occupancy at a density of 80×10⁶ cells/ml of medium (30 μl). Loaded cells at higher cell concentration show distribution in both lacunae (articular cartilage) and trabeculae (sub-chondral bone interstices). hMSCs occupy the cartilage ECM evenly (all layers).

Overall Conclusions. Feasibility of graft processing and in vitro utility has been confirmed. The primary objective of thin shell graft loading with expandable hMSCs has been met. Short-term culture demonstrates hMSC loading and initial viability in both trabeculae and cartilage lacunae.

### EXAMPLE 2

The in vitro seeding viability of combination of thin shell graft and point of service isolated bone marrow derived stem cells (BM-MSCs) is important. The example below identifies cell loading under defined vacuum seeding conditions and subsequent histological findings.

### Materials and Methods

General Thin Shell Graft Device Fabrication. Articular cartilage with underlying subchondral bone was harvested in sheets from trochlear cartilage of pigs and processed as in Example 1. Materials were stored frozen until use.

In Situ Bone Marrow-derived Mesenchymal Stem Cell (MSCs) Isolate. Bone marrow (BM) aspirates were collected from the trochlear notch of normal volunteer donors after consent. Enriched cell isolates containing MSCs were prepared using centrifugation and serum separator. Cells were harvested, enriched and seeded onto grafts within 6 hours.

### Thin Shell Graft and MSC Composite Loading

After thawing, thin shell grafts were washed three times with phosphate buffer saline as a pre-equilibration step. Test article for in vitro testing was made by with a biopsy punch producing 8 nun diameter plugs with proximal articular cartilage and distal subchondral bone ends. Cell suspension was uniformly added to plugs at 50, 150, or 300 uL per graft with normal saline used as a no cell control. Cells were loaded into the test article by six 1-minute cycles of pulse vacuum from 0 mmHg (ambient) to 550 mmHg. The four seeding groups were further stratified into two incubation groups, one at ambient pressure and the other subjected to staged vacuum at 550 mmHg vaccum for 45 minutes followed by ambient pressure for 15 minutes. After vacuum seeding and incubation, grafts were fixed with 10% neutral buffer formalin for 48 hours. Table 3 below outlines the groups and parameters tested.

TABLE 3
Example 2 Cell Loading

| | | GRAFT | | | |
|---|---|---|---|---|---|
| SPECIMEN | CELL GROUP | VOL | VOL RATIO | VACUUM | |
| ID | VOL (uL) | (mm\hat{ }3) | CELL:GRAFT | SEQUENCE | INCUBATION |
| GRP 1 | 50 | 150 | 1:3 | 550 mmHg, 6 × | 1 hr no vac |
| | | | | 1 min | |
| GRP 2 | 150 | 150 | 1:1 | 550 mmHg, 6 × | 1 hr no vac |
| | | | | 1 min | |
| GRP 3 | graft control | 150 | n/a | n/a | n/a |
| GRP 4 | cell control | n/a | n/a | n/a | n/a |
| GRP 5 | 150 | 150 | 1:1 | 550 mmHg, 6 × | 45 min vac, 15 min |
| | | | | 1 min | no vac |
| GRP 6 | 300 | 150 | 2:1 | 550 mmHg, 6 × | 45 min vac, 15 min |
| | | | | 1 min | no vac |

As shown in Table 3, ten samples were submitted for histological processing. Cartilage samples were 8 mm in diameter and nominally 3 mm thick, with a waxy hyaline side and rough subchondral bone side. Specimens were fixed in 10% neutral buffer formalin, decalcified, paraffin embedded, and step sectioned at two levels in coronal plane in 6 μm sections. Specimens were stained with hematoxylin and eosin for cell distribution analysis.

### Results

Control samples (Group 3) presented with only nomimal cell content and few cells within the trabeculae or lacunae intact. Cell content was present in the low loading group (Group 1), and increased incrementally from Group 2 to Group 5 presenting with higher cell densities in the trabeculae and columnar organized cells through the tidemark and interior cartilage. Group 6 reproducibly demonstrated the highest cell loading with some trabecular regions packed with cells and more uniform distribution of cell within the cartilage.

### EXAMPLE 3

This in vivo study describes the implant feasibility of thin articular cartilage grafts, harvested from porcine trochlear cartilage. The shell graft consists of primarily hyaline articular cartilage, with one-sided coverage of subchondral bone. The study used processed and decellularized thin shell grafts seeded in situ with point of service bone marrow cells isolates, and MSC-enriched synovial cells as the test groups controlled against fresh harvested shell grafts with viable cells as implant controls. Implantation of test and control shell grafts was performed in Yucatan pigs. The overall goal was to develop a biologically active, intact cartilage alternative to prosthetic knee replacement to treat advanced osteoarthritis.

### Materials and Methods

Pilot Study: 12-week Evaluation. Seven skeletally mature Yucatan pigs were implanted and sacrificed at 12 weeks as part of model feasibility. Three animals were implanted with fresh harvested thin shell grafts as a positive control. Two animals were implanted with shell grafts seeded with bone marrow cellular isolates. Two animals were implanted with shell grafts seeded with MSC-enriched synovial cell isolates. Evaluations included: initial cell viability; implant morphology and knee assessment at necropsy by ordinal grading; and, histology with qualitative analysis.

Study Design Overview, Test Article Allocation, and Evaluations.

TABLE 4
Study Design

| | | |
|---|---|---|
| ANIMAL # | TEST GROUP | EVALUATIONS |

Feasibility Study: 12-week Evaluation

| | | |
|---|---|---|
| FH1 | TSG-fresh | Cell viability |
| FH2 | harvest | Implant morphology and knee |
| FH3 | | assessment at necropsy with ordinal |
| SCI1 | TSG + SC-isolate | grading Qualitative histology |
| SCI2 | | |
| BMI1 | TSG + BM-isolate | |
| BMI2 | | |

### Procedures

### Test Article Preparation:

Thin Shell Graft Harvest and Processing (Test article). Thin shell grafts were harvested from trochlear cartilage of >8 month old Yorkshire pigs under controlled conditions. The grafts were sized to ovoid shape of nominally 10-mm wide by 40 mm long and 3.0 to 3.5 mm thick. Grafts were decellularized by pressurized lavage followed by hypotonic and detergent buffer incubation. Processed grafts were provided frozen for in situ formulation with cell isolates immediately before implantation.

Thin Shell Graft Harvest (Control Grafts). The control shell graft for this evaluation will emulate allogeneic osteochondral grafts and implement unprocessed shell grafts retaining viable cells. As with the test article shell graft preparation, grafts were harvested from trochlear cartilage of >8 month old Yorkshire pigs and nominally sized to ovoid shape of 10 mm wide by 40 mm long and 3.0 to 3.5 mm thick. These grafts were harvested within a week of indexed surgery and stored in DMEM solution at 4° C. to retain optimal cell viability upon implantation.

### Cell Seeding and Surgical Implantation:

In Situ Preparation of MSC Seeded Shell Grafts. In situ isolated human cells (either bone marrow or synovial fluid derived) were pulse vacuum seeded into the test article shell grafts using a cell stock concentration of 1.2×10⁶ cells per mL and 300 μL per cm² of graft material. The seeded constructs were then incubated at room temperature for 1 hour before implantation. A parallel processed sample of the synovial cell seeded constructs as those implanted were analyzed for initial cell viability by fluorescent live/dead assay and confocal microscopic analysis. Marrow isolate seeded samples were analyzed similarly with samples prepared prior to the day of implantation.

Condylar Defect Preparation and Shell Graft Placement. The surgical approach for device implantation was through standard open technique and medial para-patellar incision of the medial condyle. All animal management followed current animal care and use committee guidelines.

The defect was outlined using a pre-shaped foil template and articular cartilage removed through to subchondral bone within the outline. Cartilage within the defect was removed with a combination of rotating burr and small reciprocating saw under irrigation. Shoulders of the defect were formed square to the articular surface and defect bed morselized with a surgical awl to bleeding bone. The defect size was standardized to 3.0 to 3.5 mm in depth covering a roughly 8 mm×16 mm central portion of the anterior condyle as the knee was in flexion. An aliquot of cell isolate equal to the defect area was applied to the morselized defect bed (1.0 mL).

The pre-sized and seeded shell graft was brought to the surgical field and secured flush in the defect using resorbable anchors (ConMed/Linvatec, Smart Nail System, 1.5×16 mm) on proximal and distal third of the graft. Standard surgical closure was accomplished using interior resorbable and exterior non-resorbable suture and incision management.

### Post-Mortem Evaluations

Gross Pathology at Necropsy and ICRS Scoring. Clinical photographs of operative and contralateral knees were taken at time of necropsy and any degenerative changes in the knees recorded. Gross observations of the implant were recorded in accordance with the ICRS grading scale for arthroscopic observations resulting in a composite ordinal score for the graft.

Histology and Assessment. Two segments along the curvature of the graft were sectioned in coronal plane and include for analysis. Specimens were fixed with 10% neutral buffered formalin, decalcified using EDTA, embedded in paraffin, and sectioned at 6 μm in coronal plane with medial/lateral and superior host interface intact. Sections were taken at 2 levels for each segment and stained with H&E. trichrome, and S&O/fast green. Specimens were evaluated qualitatively for graft integration and engraftment integrity.

### Results

### Initial Cell Viability:

MSC Enriched Synovial Cell Isolate Seeded Grafts. Control grafts, processed and decellularized, exhibited only nominal cell staining in the hyaline surface and superficial zone and presented with a linear band of dead cells on the hyaline surface. The tidemark zone exhibited some live cell staining, without cells in the deeper proximal cartilage. In contrast, enriched synovial cell seeded grafts exhibited live cell staining throughout the hyaline surface and distally and packed cells associated with deeper and tidemark zones.

Marrow Cell Isolate Seeded Grafts. As with the synovial isolates, the control grafts exhibited some dead and live cell content, but limited to the exterior hyaline surface and superficial cartilage layer. In contrast, cell loaded specimens exhibited mostly live cell distribution from hyaline periphery through cartilage toward the tidemark. The subchondral side of cell treated specimens exhibited areas of densely packed cells within trabeculae, with plump cells attached to bone interstices. These results parallel findings and densities found with culture expanded BM-MSC's.

Graft Implantation. Access to the entire posterior condyle was limited without deleterious surgical excision of the meniscus and medial collateral ligament. The target area for defect creation was limited to an anterior left medial condyle surface easily accessible with the knee in flexion and soft tissue retracted medially. Rectangular defects, nominally 8 mm wide and 16 mm long, were individually sized for each condyle, with grafts sized and test fit to the defects before cell seeding. Table 5 below reviews graft test article implantation size and comments.

TABLE 5
Implant Size and Comments

| | | IMPLANT | |
|---|---|---|---|
| | | SIZE | |
| ANIMAL | TEST | (l × w) | |
| # | GROUP | mm | COMMENT |
| FH1 | TSG-fresh | 10 × 8 | 1 centrally located fixation |
| | | | anchor |
| FH2 | harvest | 10 × 6 | 2 fixation anchors |
| FH3 | | 16 × 7 | 2 fixation anchors |
| SCI1 | TSG + SC- | 16 × 8 | 2 fixation anchors |
| SCI2 | isolate | 22 × 11 | 2 fixation anchors |
| BMI1 | TSG + BM- | 15 × 8 | 2 fixation anchors |
| BMI2 | isolate | 16 × 9 | 2 fixation anchors |

Peri-Operative Findings. All animals were successfully implanted and maintained with analgesics and pain medication management for 7 days post-operatively. All animals were ambulatory by 10 days post-operatively and were not braced or cast during or after this initial recovery period.

Implant and Control Gross Pathology and Histology. Five of the seven implanted animals presented with intact and integrating grafts at 12-week sacrifice. Animals SCI2 (synovial isolate) and BMI2 (bone marrow isolate) presented with only partial or fractured grafts and general condyle degeneration. The failure mode for these implants is attributed to fixation failure, subsequent graft loosening, and not related to long-term graft performance. Considering that this animal model and graft implantation system is under development, findings from these grafts are considered non-evaluable and excluded from full analysis.

Collages incorporating gross pathology photographs and trichrome stained histology micrographs were made and analyzed. All collages showed uniform presentation of gross pathology and representative trichrome stained histology of the implanted graft and host margins at 10×, 40× and 100× magnification.

Intact Controls. Histology shows an intact hyaline cartilage layer with transition through tidemark and subchondral bone. Cellular staining in all specimen is minimal due to decalcification preparation needed for these specimens.

Fresh Harvested Graft Controls. Histology demonstrates an intact cartilage surface, devoid of fissuring, with nominal subchondral changes. Host border integration extends through the subchondral zone with surface integration represented by mixed cartilage. Extensive integration of the graft with mature remodeling extended through to the subchondral zone. The integration border presents with mixed cartilage, and graft surface organized in columnar pattern through the extra-cellular matrix.

Synovial Cell Isolate Graft. Grossly, the integrating graft presents with some peripheral margin, more pronounce at the distal pole. Histologically, graft remodeling extends deep into the subchondral zone with some focal areas of condensing fibrocartilage. Regions of marrow components are associated with the fibrocartilage. Border integration contains mixed cartilage and the graft surface has diffuse lacunae organization through the extra-cellular matrix.

Bone Marrow Cell Isolate Graft. Grossly, the integrating graft presents with a distinct peripheral margin, more pronounce laterally. Histologically, graft remodeling transitions abruptly at subchondral zone with a pin tract visible through to trabelular bone. The graft is slightly recessed and shows a mixed cartilage with underlying bone border. The graft cartilage surface has remodeled to fibrocartilage on one border and has diffuse lacunae organization through the extra-cellular matrix centrally.

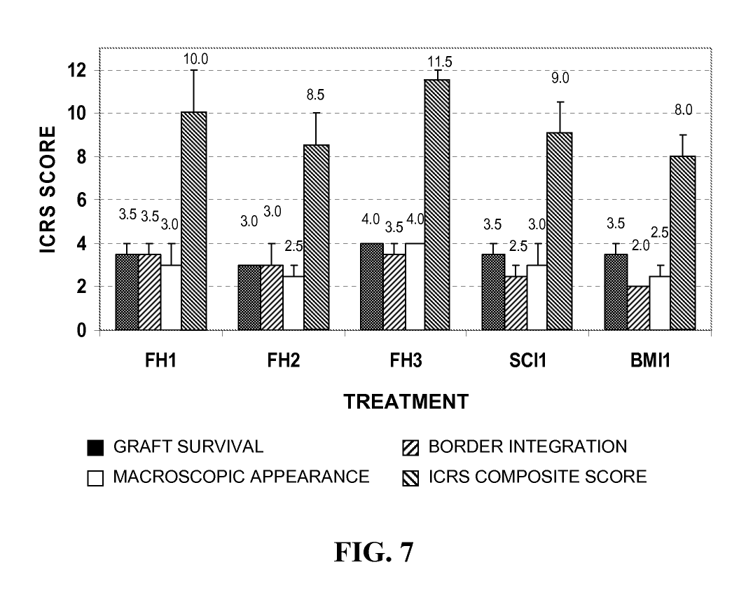

ICRS Scoring of Implants. The grafts were evaluated for gross repair using the ICRS Cartilage Repair Assessment. The ICRS assessment is optimized for osteochondral grafts and evaluates graft repair with ordinal 0-4 graded indices including: i) graft survival; ii) integration to border zone; and iii) macroscopic appearance. A composite score totaling the three indices serves a quantitative evaluation of overall graft healing. Table 6 presents the details of the assessment. Grafts were evaluated by two orthopaedic trained observers and data presented as the average of the two scorers, as shown in Table 7 and FIG. 7.

TABLE 6
ICRS Cartilage Repair Assessment
I. GRAFT SURVIVAL

| | |
|---|---|
| 4 | 100% survival of initially grafted surface |
| 3 | 75% survival of initially grafted surface |
| 2 | 50% survival of initially grafted surface |
| 1 | 25% survival of initially grafted surface |
| 0 | 0% (plugs are lost or broken) |

II. INTEGRATION TO BORDER ZONE

| | |
|---|---|
| 4 | Complete integration with surrounding cartilage |
| 3 | Demarcating border <1 mm |
| 2 | ¾ of graft integrated, ¼ with a notable border, >1 mm width |
| 1 | ½ of graft integrated with surrounding cartilage, ½ with a notable |
| | border >1 mm |
| 0 | From no contact to ¼ of graft integrated with surrounding cartilage |

III. MACROSCOPIC APPEARANCE

| | |
|---|---|
| 4 | Intact smooth surface |
| 3 | Fibrillated surface |
| 2 | Small, scattered fissures or cracks |
| 1 | Several, small or few but large fissures |
| 0 | Total degeneration of grafted area |

OVERALL REPAIR ASSESSMENT

| | | |
|---|---|---|
| Grade I | normal | 12 - P |
| Grade II | nearly normal | 11-8 P |
| Grade III | abnormal | 7-4 P |
| Grade IV | severely abnormal | 3-1 P |

TABLE 7
ICRS Cartilage Repair Assessment Scoring

| ANIMAL # | FH1 | FH2 | FH3 | SCI1 | BMI1 |
|---|---|---|---|---|---|
| Graft Survival (0-4) | 4/3 | 3/3 | 4/4 | 4/3 | 4/3 |
| Border Integration (0-4) | 4/3 | 4/2 | 4/3 | 3/2 | 2/2 |
| Macroscop. App. (0-4) | 4/2 | 3/2 | 4/4 | 4/2 | 3/2 |
| ICRS Score Obs1 | 12 | 10 | 12 | 11 | 9 |
| ICRS Score Obs2 | 8 | 7 | 11 | 8 | 7 |
| ICRS Score Ave | 10 | 8.5 | 11.5 | 9.5 | 8 |
| ICRS Grade | nearly | nearly | nearly | nearly | nearly |
| | normal | normal | normal | normal | normal |

scores on a 0-12 scale were 10, 9 and 8 for fresh harvested, synovial cell seeded and bone marrow seeded grafts respectively. Inter-observer variability was within one ordinal score for the ICRS indices. In general, the graft integration trend is supported by comparison of ICRS grade and histological findings.

### Conclusions

The viability of using thin shell grafts, both fresh harvested and cell seeded, has been shown with direct applicability toward human osteoarthritic cartilage replacement as an alternative to prosthetic knee replacement.

This example demonstrates that thin shell grafts can be processed, shaped and surgically implanted in large, full-thickness contoured defects in articular cartilage. It illustrates that cells of differing populations can be successfully seeded into cartilage constructs, and that maintaining or supplementing the biological activity of grafts can be achieved. This example further supports that grafts maintaining cells can result in mid-term cartilage replacement, and deep defect subchondral graft integration can be accomplished and is not rate limiting. Graft margin integration can be influence by fit, fixation and biologic variables, and grafts, independent of processing, maintain morphological surface integrity in mid-term evaluations.

As will be appreciated, various combinations of the features and methods described herein may be incorporated into other devices and processes according to the description herein. Accordingly, all combinations of the disclosed features and methods fall within the scope of this disclosure.

Although this invention has been described in terms of certain embodiments, other embodiments that are apparent to those of ordinary skill in the art, including embodiments which do not provide all of the benefits and features set forth herein, are also within the scope of this disclosure. Accordingly, the scope of the present invention is defined only by reference to the appended claims.

1. A graft for implantation in an articular cartilage defect in a bearing region of a articular surface of a joint of a patient, wherein the articular cartilage defect is characterized by a base surface disposed about a defect axis extending substantially normal to the articular surface at the defect, and defined by a defect base periphery and having a lateral surface extending in the direction of the graft axis from the defect base periphery with monotonically increasing radii with respect to the defect axis, comprising:
an intact tissue block:
A. extending along a graft axis from an outer surface at an outer end to an inner surface at an inner end, wherein the outer surface is bounded by an outer end periphery and extends transverse to the graft axis at the outer end, and the inner surface is bounded by an inner end periphery and extends transverse to the graft axis at the inner end,
B. having a lateral surface extending along and about the graft axis from the outer end periphery to the inner end periphery,
C. including at the outer end, hyaline cartilage extending from the outer surface and in the direction of the graft axis, toward the inner end, and
D. including at the inner end, subchondral bone extending from the inner surface and in the direction of the graft axis, toward the outer end,
wherein the outer surface as defined by the outer end periphery, has a shape adapted to overlie and extend beyond the bearing region of the articular surface of a joint when the graft axis is substantially coaxial with the defect axis,
wherein the inner surface as defined by the inner end periphery, has a shape adapted to overlie and is coextensive with the base surface of the defect when the graft axis is substantially coaxial with the defect axis,
wherein the lateral surface of the graft is substantially complementary to the lateral surface of the defect and
wherein the maximum thickness T of the graft in the direction of the graft axis, is such that when implanted, the graft is resistant to fracture under anatomical load of the patient.

2. A graft according to claim 1 wherein the patient is a human-, the tissue block is from a human, and T is in the approximate range 2.5-12.0 mm.

3. A graft according to claim 1 wherein the tissue block is substantially devoid of cellular activity.

4. A graft according to claim 1 wherein the tissue block is characterized by reduced cellular activity.

5. A graft according to claim 1 wherein the subchondral bone of the tissue block is substantially devoid of cells.

6. A graft according to claim 1 wherein the tissue block is characterized by near normal cellular activity.

7. A graft according to claim 1 wherein the tissue block is characterized by reduced cellular activity pursuant to a treatment from the group consisting of freeze/thaw cycling, hypotonic/hypertonic solutions, ionic/anionic detergents, compressed CO₂ gas facilitated lavage, or combinations thereof.

8. A graft according to claim 1 wherein the tissue block is sterilized by a sterilization process including one or more of ionizing radiation or supercritical CO₂ sterilization processes.

9. A graft according to claim 1 wherein the tissue block is sterilized by supercritical CO₂ sterilization.

10. A graft according to claim 1 wherein the tissue block is sterilized to effect a bioburden reduction of at least 10⁶.

11. A graft according to claim 1 wherein the subchondral bone of the tissue block is infused with exogenous cells.

12. A graft according to claim 1 wherein the subchondral bone of the tissue block is vacuum-infused with exogenous cells.

13. A graft according to claim 1 wherein the subchondral bone of the tissue block is infused with one or more bioactive agents to enhance healing.

14. A graft according to claim 1 wherein the subchondral bone of the tissue block is vacuum-infused with one or more bioactive agents to enhance healing.

15. A graft according to claim 1 wherein the tissue block includes distributed therein, a cell population including one or more cells from the group consisting of adult or embryonic mesenchymal stem cells, embryonic stem cells, fibroblasts, chorndrocytes, chondroblasts, pro-chondroblasts, osteocytes, synoviocytes, osteoclasts, pro-osteoblasts, monocytes, pro-cardiomyocytes, pericytes, cardiomyoblasts, cardiomyocytes, myocytes or combinations thereof.

16. A graft according to claim 15 wherein the cell population includes cells from bone marrow.

17. A graft according to claim 15 wherein the cell population includes cells from adipose tissue.

18. A graft according to claim 15 wherein the cell population includes cells from plasma derived fractions of autologous blood.

19. A graft according to claim 15 wherein at least a portion of the cell population is vacuum-infused into the tissue block.

20. A graft according to claim 15 wherein a loading ratio of cells of the population in a volume of cells to volume of graft, ranges from 1:3 to 3:1.

21. A graft according to claim 1 wherein the tissue block includes distributed therein, one or more bioactive agents.

22. A graft according to claim 21 wherein the bioactive agents include one or more from the group consisting of fibroblast growth factors, epidermal growth factors, kertinocyte growth factors, vascular endothelial growth factors, platelet derived growth factors, transforming growth factors, bone morphogenic proteins, parathyroid hormone, calcitonin, prostaglandins, ascorbic acid, and combinations thereof.

23. A graft according to claim 22 wherein a loading ratio of cells of bioactive agents in a volume of cells to volume of graft, ranges from 1:3 to 3:1.

24. A graft according to claim 1 wherein the tissue block is from an animal from the group consisting of porcine, bovine, equine or ovine animals.

25. A graft according to claim 1 wherein the tissue block is from an animal from the group consisting of porcine, bovine, equine or ovine animals pursuant to de-antigenation by removal of alpha-galactosyl epitopes with glycosidase.

26. A graft according to claim 1 wherein the tissue block is from a human.

27. A graft according to claim 1 wherein the articular surface is a joint from the group consisting of knee, jaw, shoulder, elbow and hip.

28. A method for infusing a cell population or one or more bioactive agents into a tissue block extending from a first end to a second end opposite thereto, and including at the first end, hyaline cartilage extending from the first end and toward the second end, and including at the second end, subchondral bone extending from the second end toward the first end, comprising the steps of:
A. positioning the cell population or bioactive agents onto at least on surface of the tissue block;
B. applying a pressure gradient to the tissue block; having the cell population or bioactive agents thereon;
wherein the application of the pressure gradient comprises the steps of:
applying a pulsed vacuum sequence to the tissue block having the cell population or bioactive agents thereon, cycling n times between approximately 0 mmHg (ambient) and approximately 750 mmHg, for durations m minutes, where n and m are integers.

29. The method of claim 28 wherein the cycles are uniform from cycle to cycle, and m is in the range 3-10 cycles and n is in the range 1 to 3 minutes.

30. The method of claim 29 wherein, following the application of the pulsed vacuum sequence to the tissue block, the graft is incubated under vacuum for a period T₀ at a pressure P.

31. The method of claim 30 wherein T₀ is in the range 45-120 minutes and P is in the range 200-750 mmHg.

32. The method of claim 30 wherein T₀ is in the range 45-120 minutes and P is in the range 300-550 mmHg.

33. A method for preparing a human allograft or xenograft for implantation in an articular cartilage defect, comprising the steps of:
A. asceptically harvesting a graft including an intact tissue block from a host, wherein the tissue block:
a. extends along a graft axis from an outer surface at an outer end to an inner surface at an inner end, wherein the outer surface is bounded by an outer end periphery and extends transverse to the graft axis at the outer end, and the inner surface is bounded by an inner end periphery and extends transverse to the graft axis at the inner end,
b. has a lateral surface extending along and about the graft axis from the outer end periphery to the inner end periphery,
c. has at the outer end, hyaline cartilage extending from the outer surface and in the direction of the graft axis, toward the inner end, and
d. has at the inner end, subchondral bone extending from the inner surface and in the direction of the graft axis, toward the outer end,
B. decellularizing the graft;
C. de-antigenizing the graft;
D. sterilizing the graft; and
E. infusing a cell population or one or more bioactive agents into a tissue block if the graft.

34. A method for implanting a graft in an articular cartilage defect in a bearing region of a articular surface of a joint of a patient, comprising the steps of:
A. preparing the articular cartilage defect whereby it is characterized by a base surface disposed about a defect axis extending substantially normal to the articular surface at the defect, and defined by a defect base periphery and having a lateral surface extending in the direction of the graft axis from the defect base periphery with monotonically increasing radii with respect to the defect axis,
B. preparing a graft in accordance with claim 33 and whereby:
a. the outer surface as defined by the outer end periphery, has a shape adapted to overlie and extend beyond the bearing region of the articular surface of a joint when the graft axis is substantially coaxial with the defect axis,
b. the inner surface as defined by the inner end periphery, has a shape adapted to overlie and is coextensive with the base surface of the defect when the graft axis is substantially coaxial with the defect axis,
c. the lateral surface of the graft is substantially complementary to the lateral surface of the defect, and
d. the maximum thickness T of the graft in the direction of the graft axis, is such that when implanted, the graft is resistant to fracture under anatomical load of the patient;
C. preparing the lateral surface of the defect for receipt of the graft by the step of morselizing the lateral wall and the base surface through a subchondral plate underlying the defect;
D. applying the graft to the defect whereby the lateral surface of the graft is in intimate contact with the lateral surface of the defect; and
E. attaching the graft to the base surface of the defect.
